(19) United States
(12) Patent Application Publication
(10) Pub. No.: US 2004/0132141 A1
(43) Pub. Date: Jul. 8, 2004

(54) Selecting therapeutic human monoclonal antibodies from disease-specific libraries

(76) Inventor: Kimon Angelides, Houston, TX (US)

Correspondence Address: Eric P. Mirabel, Suite 100, 35 Technology Drive, Warren, NJ, 07059, US

(21) Appl. No.: 10/651,333

(22) Filed: Aug. 28, 2003

Related U.S. Application Data
(60) Provisional application No. 60/406,510, filed on Aug. 28, 2002.
(60) Provisional application No. 60/406,456, filed on Aug. 28, 2002.
(60) Provisional application No. 60/406,457, filed on Aug. 28, 2002.
(60) Provisional application No. 60/408,215, filed on Sep. 4, 2002.
(60) Provisional application No. 60/408,947, filed on Sep. 6, 2002.
(60) Provisional application No. 60/408,948, filed on Sep. 6, 2002.

(51) Int. Cl. G01N 33/53; C12P 21/04

(52) U.S. Cl. 435/70.21; 436/548

(57) ABSTRACT

The invention relates to a method of generating human monoclonal antibodies suitable as therapeutic candidates from a pool of human peripheral blood lymphocytes extracted from a number of individuals who have all been exposed to the same disease antigen. Peripheral blood lymphocytes extracted from said individuals are immortalized, and a high throughput functional screening assay of the immortalized lymphocytes against target cells associated with or affected by the disease is peformed, in which the target cells are coated onto microbeads and placed in wells formed in one of the ends of a bundle of optical fibers, and wherein changes to the target cells resulting from the assay can be optically detected at the opposite end of the bundle. The immortalized lymphocytes producing human antibodies having specific, desired effects on the target cells, as determined by the assay are selected, grown, and the antibodies are further characterized, in order to select therapeutic candidate antibodies. Also described is a method of screening antibodies against peptide libraries, where the peptides represent or encompass known disease epitopes.

### PRIORITY CLAIM

Priority is hereby claimed to U.S. Provisional Application Serial Nos. 60/406,510; 60/406,456; 60/406,457 (all of which were filed on Aug. 28, 2002), to Serial No. 60/408,215, filed Sep. 4, 2002, and to Serial Nos. 60/408,947; 60/408,948, both filed on Sep. 6, 2002.

### BACKGROUND OF THE INVENTION

1. Isolating Human Antibodies From Diseased Patient Pools

An immune system defends against invasion by microorganisms by producing

antibodies, proteins which can complex with the invading microorganisms and target them for destruction or removal. Any foreign molecule of sufficient size can act as a stimulus for antibody production. B lymphocytes, the cells that actually produce antibodies, recognize and respond to an antigen by reproducing or cloning themselves and then producing antibodies specific to that foreign invader. In a viral infection, because many viruses are adept at mutation, there is a competition between the immune response and the virus, with each adapting to mutations made by the other in an effort to achieve victory. A successful outcome for the host requires that the virus can no longer evade the host defense. This may require a series of antibodies produced by the B cell such that each move and countermove ends in an immunological checkmate, where the “matured” antibody produced is sufficiently refined to ward off all (or most) subsequent attacks of the same or similar invaders. Once the foreign invader is destroyed, the production of the refined antibodies by B cell lymphocytes subsides and the B cell returns to a resting state, although it retains a genetic memory of the foreign invader and can respond effectively on a subsequent attack.

Recent technological advances have made it possible to isolate and cultivate a single clone of lymphocytes to obtain a virtually unlimited supply of antibodies specific to particular disease targets. These antibodies, known as “monoclonal antibodies” because they arise from a single clone of lymphocytes, are produced by hybridoma technology. Conventional hybridoma technology takes advantage of the fusion of an immortal cell, in most cases a particular cancer cell known as a myeloma cell, with spleen cells from a mouse that has been injected or immunized with an antigen. These fusions are isolated by transferring them to a growth fluid that kills off the unfused cancer cells. The unfused spleen cells die off by themselves. The fused hybrid spleen and myeloma cells, called hybridomas, produce antibodies to the antigen initially injected into the mouse. The single hybridoma or hybridomas secreting antibody into the medium are then screened and those with the desired characteristics are selected. Each hybridoma then reproduces itself and these identical hybridomas each produce identical monoclonal antibodies having the same affinity and specificity. In this way, a virtually unlimited supply of identical antibodies is created, directed to only antigens used for screening.

Accordingly, if rather than starting with an immunized animal, one captures the monoclonal antibody repertoire of B cells produced by an individual's immune system, one can use the benefits the human immune systems' “adaptive immunity.” Adaptive immunity refers to ability of the human immune system to improve as it repeatedly encounters various diseases or antigens. Through evolution, the human immune system has developed a significant library of antibody responses that cannot be duplicated in the monoclonal antibody panels created in mice, by immunization of animal host organisms, or by other methods used to create monoclonal antibodies, including recombinant phage display.

In the case of an individual suffering from an infectious disease or a tumor, he/she will have a repertoire of B cells producing antibodies to the infectious agent or the tumor. Human antibodies have major advantages over their murine or animal counterparts. They can be designed to induce effector functions, including cell killing, and they do not induce human anti-mouse (“HAMA”) or other allergic or undesirable responses. However, it can be difficult to isolate human monoclonal antibodies with therapeutically useful affinities and other therapeutic properties from diseased individuals, possibly because the desired antibodies are often present in a low titer. It is believed, therefore, that administering human monoclonal antibodies of the same specificity as some that are generated by such patients may be a viable method to treat tumors or infectious diseases. This is known as passive immunization.

To find therapeutically useful human antibodies, it is believed that the more patients one isolates protective antibodies from, which provides a larger pool of antibodies to screen, the better the resulting monoclonal antibody candidates will be. That is, one is more likely to find antibodies which have improved cell killing ability, increased affinity, increased specificity (they do not target or kill any other cells) and other desirable properties.

A further problem that has been discussed relating to isolation of protective antibodies, is that antibodies may only protect the patient it was isolated from, or, in the alternative, may protect other patients and not the patient it was isolated from. This can be true even where the disease cells appear to be identical or nearly identical from patient-to-patient. Therefore, another advantage of screening for antibodies from a large number of patients is that one is more likely to isolate cross-protective antibodies, useful in large numbers of patients having a particular disease or condition.

If one starts with a pool of lymphocytes from a patient population all having the same disease, and one wishes to find optimal human antibodies for therapy for that disease, the first level of screening is to isolate cells expressing antibodies which are likely to be therapeutic. For example, if the patients all have a particular tumor, one screens for cells expressing antibodies capable of killing those tumor cells. With such a screen, one isolates a disease-specific pool or library of antibody expressing cells. To achieve such a pool or library, however, one needs a high throughput screening method that screens cells based on function, ie., a high throughput functional assay. Such a screening method has not previously been available.

Another phenomenon which has been discussed in the field of passive immunization is that it may require multiple antibodies, all acting in combination or synergistically, to have a therapeutic effect, such as killing a tumor or infected cell. It has been difficult previously to screen for multiple antibodies which, in combination, have such a therapeutic effect, especially where a pool of antibody-expressing cells are taken from many patients and there are a large number of combinations of antibodies possible. Again, a high throughput method of screening for therapeutic antibodies which are effective, or more effective, in combination is needed.

2. Generating Human Monoclonal Antibodies

To generate a library or pool of immortal cells expressing antibodies useful in therapy of a particular disease, one well-established method is to transform human peripheral blood lymphocytes from a patient with the disease of interest with Epstein Barr virus, or EBV, as described, for example in Gomy, M. K. et al., Proc. Nat'l. Acad. Sci. USA 86:1624-1628 (1989). Generation of human antibodies from primed donors has also been performed by stimulation with CD40, resulting in expansion of human B cells, Banchereau et al., F. Science (1991), 251:70, Zhang et al., J. Immunol. (1990), 144, 2955-2960, Tohma et al, J. Immunol. (1991), 146:2544-2552, or by an extra in vitro booster step primer to immortalization. Chaudhuri et al., Cancer Supplement (1994), 73, 1098-1104. The B cells resulting from either method are then fused with a myeloma cell, forming immortal monoclonal antibody-producing hybridomas. See, e.g., U.S. Pat. No. 4,897,466, incorporated by reference.

One would now have a large pool of hybridomas, some of which produce antibodies to the disease antigen of interest. The next step is to screen this pool for those cells that express antibodies suitable for disease therapy.

### SUMMARY OF THE INVENTION

The invention relates to a method of generating human monoclonal antibodies suitable as therapeutic candidates from a pool of human peripheral blood lymphocytes extracted from a number of individuals who have all been exposed to the same disease antigen. Starting from a pool of patients which all have a particular disease suitable for treatment with passive immunization therapy, including tumor-related diseases and infectious diseases, one extracts from each patient's serum peripheral B lymphocytes. The B lymphocytes are then immortalized by well known techniques, including EBV transformation followed by cell fusion, or direct fusion with a cell line, human or otherwise, which does not require prior transformation, e.g., the cell line described in International Patent Application No. WO 02/04607, by A. Karpas. The immortalized cells are screened using a high throughput functional assay against target cells associated with or affected by the disease, in which the target cells are coated onto microbeads and placed in wells formed in one of the ends of a bundle of optical fibers, and wherein changes to the target cells resulting from the assay can be optically detected at the opposite end of the bundle. The immortalized lymphocytes producing human antibodies having specific, desired effects on the target cells, as determined by the assay are selected, grown, and the antibodies are further characterized, in order to select therapeutic candidate antibodies. The candidate cell lines can be further characterized for other desired properties, including high affinity, cross-protection, lack of cross-reactivity with other antigens, and others. Moreover, by using a screening method in which multiple antibody-producing cells are monitored for their effectiveness against target cells or for desired properties, combinations of antibodies which act additively or synergistically can be isolated.

Additionally, described below is a method of isolating therapeutic human monoclonal antibodies useful in therapy, by screening a library of human monoclonal antibodies against a library of peptides, and then isolating those antibodies which interact with peptides in the library known to represent or encompass disease-associated epitopes.

Particular methods of high throughput screening and further examples of making and using the invention are discussed further below.

### BRIEF DESCRIPTION OF THE DRAWINGS

FIG. 1 is a flow diagram schematically illustrating certain of the various steps in the selection process of the invention.

FIG. 2 is a flow diagram schematically illustrating various steps in the selection process of the invention used to find combinations of human antibodies which act in an additive or synergistic manner.

FIG. 3 is a flow diagram illustrating production of human monoclonal antibodies using screening as set forth herein.

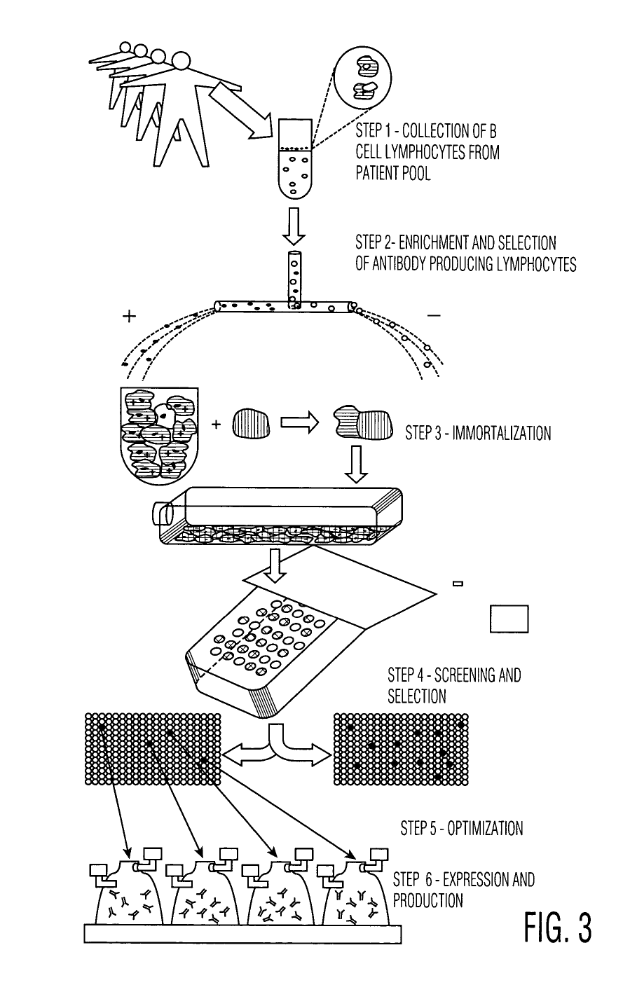

FIG. 4 depicts cells (33, 35) coated on microbeads (37) and positioned in the ends of a bundle of microfibers (31).

FIG. 5 depicts the operation of and results from an optical fiber device used in antibody screening, where a laser 41 illuminates a dichroic mirror 43 and illuminates a bundle of microfibers 31, where the ends of the fibers are in a well 45, and the signals from reactions in the well 45 are transmitted and displayed in an array 47.

### DETAILED DESCRIPTION OF THE INVENTION

A. High-Throughput Screening by FACS A key step in the process is the ability to do a high throughput screening based on antibody function. Such a screening is described in International Application No. WO 01/59429; U.S. Application Publication Nos. 2002170365; 2003013201 (Larry Sklar et al., assigned to the University of New Mexico, incorporated by reference), which utilizes an improved flow cytometer device and system. It is an improved system for high throughput cell screening of multiple samples, in which successive samples that are to be analyzed are taken from a well or other source. Each successive sample is separated with a gas before analysis by flow cytometry separation of fluorescent cells, also known as fluorescence activated cell sorting (“FACS”). FACS allows efficient selection of particular cells from large numbers of cells, based on selected characteristics, including, for example, whether a particular antibody can bind to them. Where an antibody does so, the cell will fluoresce, and the flow cytometer separates the fluorescing cells from the others in the sample.

This system keeps track of the time at which each sample is analyzed, which allows one to trace back to the source well any fluorescent cells, all in a higher throughput manner than was possible before. While other systems allowing one to trace fluorescent cells to the source are known, all provide fewer samples analyzed per unit time.

In the present invention, one of the functional assays which can be used focuses on cytotoxic activity toward cancerous cells as an end point. A live/dead assay kit, for example the LIVE/DEAD.RTM. Viability/Cytotoxicity Assay Kit (L-3224) by Molecular Probes, Inc. of Eugene, Oreg., is utilized. The Molecular Probes kit provides a two-color fluorescence cell viability assay based on the simultaneous determination of live and dead cells with two probes that measure two recognized parameters of cell viability, intracellular esterase activity and plasma membrane integrity. The assay principles are general and applicable to most eukaryotic cell types, including adherent cells and certain tissues. Modifications of the technique and dyes used can make the assay applicable to bacteria or yeast.

One way to establish a reporter which can be identified by the flow cytometer is to load the cells one is interested in killing in the functional assay (infected cells or tumor cells) with a fluorescent dye, such as propidium iodide or EthD-1, which enters cells with damaged membranes and undergoes a 40-fold enhancement of fluorescence upon binding to nucleic acids, thereby producing a bright red fluorescence in dead cells (ex/em.about.495 nm/.about.635 nm). EthD-1 is excluded by the intact plasma membrane of live cells. Another suitable marker is Almar Blue, which fluoresces if the cell is active. Alternatively, one can use the two color reporter system available from Molecular Probes, Inc. of Eugene, Oreg. The cell-permeant esterase substrate calcein AM is nonfluorescent until converted by enzymatic activity to highly fluorescent calcein, which is retained within live cells and imparts an intense green fluorescence. Ethidium homodimer-1 undergoes a fluorescence enhancement upon binding nucleic acids, producing a bright red fluorescence. This dye is excluded from cells that have intact plasma membranes but is readily able to enter dead cells. Thus, live cells fluoresce green, while dead cells fluoresce red.

The third key feature in the screening process is determination of specificity. To a certain extent specificity (ie., binding to the target cell and only to the target cell) is related to affinity. An example of this is the situation where an antigen has very limited tissue distribution. The affinity of the antibody determines specificity of the antibody. The higher the affinity the more tissue specific the antibody, because an antibody with low affinity may bind to cells other than the one of interest. Therefore, to address the specificity issue, the antibodies should be screened simultaneously against a variety of cells. In the examples below hybridoma supernatants (representing the earliest stages of monoclonal antibody development), are tested against a number of cell lines to establish specificity as well as activity.

Other fluorescent reporters can indicate if the antibody affects other cell functions, for example, intra-cellular signaling. Fluo-3 can indicate changes in the cell surface receptors that end up in calcium signals, which can indicate that the antibody is affecting one or more of G protein activation, phosphatidyl inositol signaling, or ion channels. Phosphatidyl inositol signaling can be indicated by phosphodiesterase substrates, including several unique fluorescent phosphatidyl inositol derivatives. Molecular Probes has available several reagents for studying Ca²⁺ regulation in live cells. Fluorescent nucleotides, including analogs of ATP, ADP, AMPPNP, GTP, GDP, GTP-γ-S and GMPPNP can be used, and the GTP analogs may be particularly useful in the assay of G-protein-coupled receptors. Protein-complementation assays of the JAK-STAT pathway (related to apoptosis) can be reported by a protein-protein interaction, using the reconstitution of catalytic activity of β-galactosidase, dihydrofolate reductase, or any such enzyme that is able to cleave or form a bond of a substrate, fluorescent or otherwise. Tyrosine kinase activity can also be measured by this method which is indicative of growth factor signaling.

The fluorescent dyes are added to the cells by, for example, incubating the cells with the dye. The cells may be rinsed to wash excess dye from the outer surface of the cells. Samples of these cells are then either placed into wells of a microtiter plate or mixed with antibody samples from hybridomas directly on line. The wells may also are loaded with the human antibody-producing cells taken from the diseased subjects, e.g., hybridomas or EBV-transformed B cells.

Before loading into the plate, the antibody-producing cells are subject to limiting dilution. This means that samples including such cells are diluted to the point where probability dictates that there will be only one cell per well. In this way, after identification of the wells producing antibody of interest, those wells will be likely to contain cells producing that antibody; but only those cells and not other antibody-producing cells.

Antibodies in any well which affect the cells in the well, e.g., by inducing antibody dependent cellular cytotoxicity (“ADCC”), will bind to a target cell in the well, kill it, and produce fluorescence in reporters which fluoresce on cell death. A flow cytometry system capable of allowing analysis of successive samples from wells and determination of the wells producing a reactive antibody, including those described in International Application No. WO 01/59429; U.S. Application Publication Nos. 2002170365 and 2003013201, is used to select reactive wells. The killed fluorescent cells are separated in the flow cytometer, and their source well is determined. The antibody-producing cells in the source well can then be grown, subcloned, and the antibodies further characterized and analyzed to determine their suitability for use as therapeutic candidates. Antibodies having other effects on cells and signaling pathways can be similarly monitored.

Alternatively, rather than loading the cells one is trying to kill with dye, one could use a novel system of micro-encapsulation of the cells. Suitable microcapsules include those disclosed in U.S. Pat. No. 6,586,176 (incorporated by reference) which describes encapsulation of nucleotides. In this application, one would add target cells to the wells of a microtiter plate containing human antibody-producing cells (following a limiting dilution) and then perform the encapsulation to capture both target cells and antibodies in the microcapsules. In this system, fluorescent reporters, or other reporters, within the microcapsule membrane would be activated by hydrolases released by killed cells within certain of the microcapsules; or other cellular factors related to cell signaling or live cells, as selected, could be activated upon monitoring of other cell functions. Those microcapsules would be the ones containing antibodies initiating the action under study. These microcapsules would then be separated by flow cytometry, using one of the preferred systems described above, and the source well that contains the antibodies and cells within the fluorescent microcapsules identified. Again, the antibody-producing cells in those wells would be grown, subcloned and the antibodies further characterized and studied to determine their suitability as therapeutic candidates.

The reporters in the microcapsule are imbedded in the matrix which forms the microcapsule shell. A suitable fluorescent reporter is fluorescein diacetate. Agar gel is suitable for a matrix material, as are others, for example, agarose, aiginate, carrageenan, polyacrylamide, or poly-L-lactic acid. The reporter will fluoresce upon activation by release of intracellular enzymes, for example, hydrolases inside the capsule which are released when cell-killing antibodies in the microcapsule bind the target cells and initiate cell lysis through ADCC.

To access the cells producing cell-killing antibodies, the microcapsules in the source wells can be isolated and then heated to lyse them and cause release of the antibody-producing cells.

The advantages of this micro-encapsulation system include the fact that because one is working inside a micro-capsule environment, very small volumes of antibody, as low as femtoliter quantities, are sufficient to induce the capsule to fluoresce. This means that cells which produce very small quantities of antibodies, or cells which are present themselves in a well in a very low quantity in a well, can be detected. They might otherwise fail to be detected if one was not using a micro-encapsulation process. Such cells might actually be the ones producing antibodies optimal for cell killing applications.

As a further advantage, because one can use small volumes of cells in the microcapsule, one only needs very small amounts of antibody in a sample well to induce fluorescence. It is desirable to have as many screening procedures as possible in the making of antibodies, as this leads to more specific antibodies that are more likely to be therapeutically useful. Accordingly, people have suggested pre-screening of the B cells producing antibodies before fusion or transformation to produce hybridomas, and then further screening of the hybridomas. One problem encountered with pre-screening of B cells is that they are unstable and short-lived. Thus, if they are limit diluted and placed in wells, they are so unstable that they can die before enough time passes for them to produce detectable amounts of antibodies.

However, with the system of the present invention, because such small volumes of antibodies can induce fluorescence of the microcapsule, it is possible to pre-screen B cells. Very small titers of the B cells will produce after a short time in a well (for example, a few hours rather than several days as is normally required), sufficient quantities of antibody to kill enough cells to induce fluorescence. The B cells in the fluorescent microcapsules (or in the wells from which the fluorescent microcapsules were selected) can then be fused or transformed to hybridomas, and further screened to select the optimal antibody-producing cells and antibodies.

An alternative to microencapsulation, or other methods of reporting changes in the target cells, is to use two-color fluorescence correlation spectroscopy as disclosed in U.S. Pat. No. 6,200,818, incorporated by reference. With this technique, one would label the target cells with two fluorescent markers, and then illuminate the target cells with a laser in order to determine differences in fluorescence of the markers, which is correlated with known data to determine whether there was cell killing or other interaction between the target cells and the antibodies one is screening. Antibody-producing cells from the wells in a plate which contain antibodies with the desired properties can then be selected for further characterization and analysis.

B. High-Throughput Screening Using Optical Fibers and Microbeads

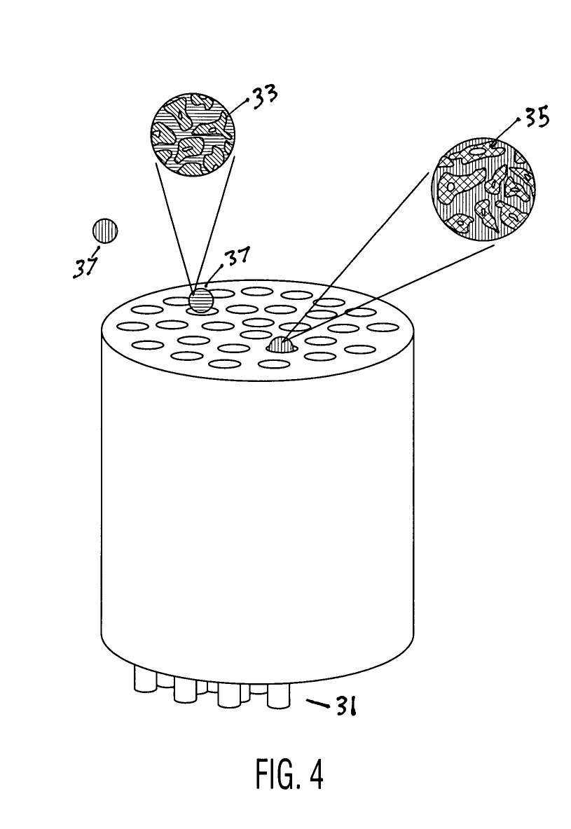

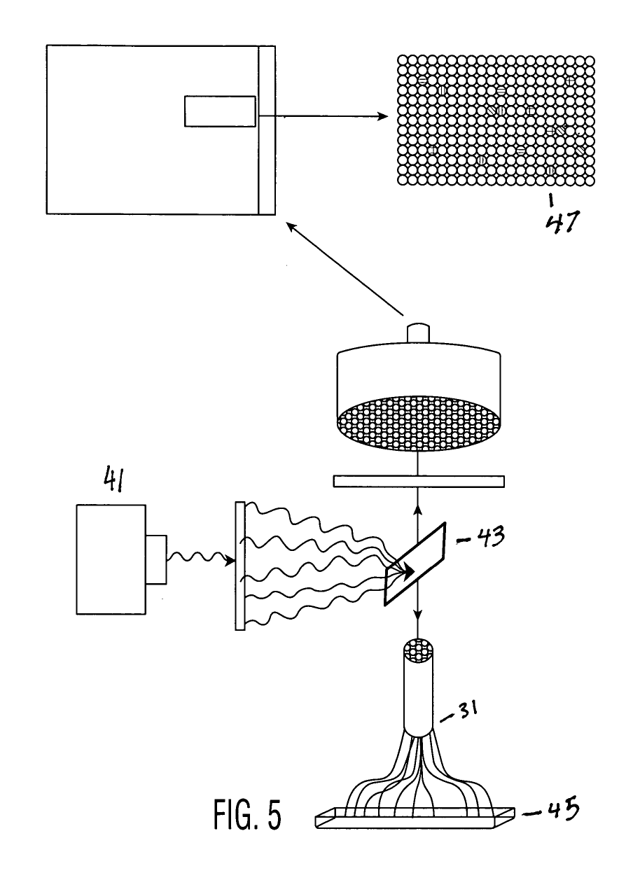

The methods of the invention preferably include an optical fiber array for determining and recording the results of essentially simultaneous assays performed on cells located at one end of the array. Each fiber in the array has a well etched into one end of it. Each well is designed to contain within it a microbead. Each microbead is coated with cells. See FIG. 4. Responses of the cells on the microbeads in the assays are monitored by reporting them to the distal end of the fibers, and recording them there. The monitoring and reporting is accomplished with a reporter system which responds to light excitation, e.g., a fluorescence marker which fluoresces when illuminated by a laser. The fluorescence marker can either be a fluorescent dye loaded into the cells, a fluorescent dye coated on the microbeads and in contact with the cells, a reporter marker which responds by fluorescing when the cell responds to stimuli and expresses or secretes a particular protein, or other systems which can accomplish the same types of reporting. The fluorescence is detected at the distal end of the fibers and recorded, e.g., with a charge coupled device or similar optical recording device for detecting photons, which generates an array of data points, with each representing the results of one particular assay on one type of cell. See FIG. 5.

In a first embodiment, each microbead is coated with several cells which are all of the same type and all representative of a disease state. For example, all beads can be coated with tumor cells or infected cells. The cells of each bead (or each bead itself) are all associated with one particular assay, but different beads can be each associated with one of several different assays. Where different beads are associated with different assays, and each bead is at the end of one fiber in an array, the outcome of any particular assay can be separately recorded at the distal end of the fiber as a point in an array. It is therefore possible to assay a library of antibodies and record the effect discrete antibodies in the library have on the cells on discrete beads, as determined by several assays, each associated with one bead, which are all performed simultaneously.

The significant advantage of using beads coated with several cells is that the effect of an antibody in the assay associated with any particular bead is amplified. If one or more of the cells carried on a bead are affected by an antibody, this will be detected by the assay and reported by a fluorescence change and recorded. If one was using only one cell per fiber well, false negatives are more likely because of failure of the target antigen on the cell surface to come into contact with a targeting antibody; or, even if there is contact, antibody binding by only a few antibodies may fail to initiate a recognizable change in the cell due to differences in affinity of the antibodies, or differences in cell signaling functionality among different cells of the same type. Using several cells per bead provides amplification of signal and lessens the likelihood of false negatives.

An advantage of using a fiber array, is that each array can have a multitude of fibers (from 5,000 to 50,000 fibers per array can readily be achieved). Because the number of assays is more limited, more than one assay is likely to be associated with a particular array. This provides considerable redundancy for each of the assays, so that it is almost certain that more than one microbead/target cell/assay combination is presented to each of the assay plate wells. A failure to register by any one (or even several) of the assays will be less likely to be recorded as a false negative for the antibodies in the assay plate well where such failure occurred.

The devices discussed herein are well-suited for monitoring of antibody-producing cells which have been limit diluted and placed into the wells of a microtiter assay plate. The device will preferably have a series of arrays designed such that one member in each array is aligned with each well in a microtiter plate, so as to allow simultaneously assaying and monitoring of one entire plate per pass. Alternatively, a single array, or several arrays, can be arranged in a pattern so that a multitude of fibers extend into each well in a multi-well assay plate. This can be accomplished by extending the ends of the fibers through a plate in the correct pattern to match the wells of the assay plate. The plate is then placed on top of the assay plate, so that the fiber ends enter the assay plate wells. Provided that the total number of assays associated with the array is substantially less than the number of fibers in each well, one can be assured that each assay will be carried out in each assay plate well, and there will be redundancy of assays.

The preferred assays include functional assays, which determine the effect that a human antibody being screened has on the function of a cell. The assays can be used to determine any of a number of cell function, including but not limited to: (i) cytotoxic activity toward cancerous cells; (ii) intra-cellular signaling, including G protein activation, phosphatidyl inositol signaling, or ion channel effects; (iii) Ca²⁺ regulation in live cells; (iv) effects on the JAK-STAT pathway (related to apoptosis); and (v) effects on tyrosine kinase activity, which is indicative of growth factor signaling. Simultaneously with a determination of function, assays can be included to determine antibody binding (a conventional enzyme-linked immunoadsorbant assay, “ELISA”), or to determine specificity, i.e., that it binds only to the target cells and not to other cell or tissues.

If desired, one could also perform screenings of different types of cells, or different subpopulations of cells, using the device. One method to screen different cell types is by performing a sequential screening, first with one cell type coated on the beads, which are then assayed for antibody reactivity, light excited and the outcomes recorded; and then with another cell type on the beads, which are again assayed, excited and recorded. In the alternative, a microbead(s) can be coated with a plurality of different cell types, with each particular cell type encoded so that it can be identified in the array. Either of these arrangements of cells and microbeads allows assays for the effect of the antibodies being screened on different cell types to be performed and recorded in one pass-through.

Coating different cell types on the same microbead, or having more than one type of assay associated with each microbead, allows one to effectively multiply the number of assays which can be conducted by each array. The reporters for the assays can be selected to indicate the results of the different assays. In one example, some beads could be coated with tumor cells and others with non-tumor cells of the same cellular type as the tumor cells. With such a system, one can simultaneously monitor the effect that an antibody has on the tumor cell and the healthy cell, and its specificity for tumor cells. The encoded bead/cell arrangement provides for an increase in throughput over sequential assaying of different cell types.

As an additional refinement to the screening process using the optical fiber device, one could combine it with fluorescence correlation spectroscopy (FCS) or high throughput FACS, including the microencapsulation techniques described above, if desired. For example, high throughput FACS or FCS could be done on the cell population first to determine cells which produce human antibodies of unknown specificity (i.e., and antibody-producing cells would be selected), and then the candidates could be further screened for desired functional characteristics and other characteristics using the optical fiber system. As a further alternative, the initial screening could be done using a microfabricated device for sorting cells, as described in U.S. Patent Application Publication No. 20020005354 (incorporated by reference), which allows high throughput cell sorting by a disposable device. Using this device, a first cell-sorting step could be to screen out the antibody-producing cells from the population of hybridomas. From there, they could be subjected to functional screening by one of the methods described above.

Optionally, to potentially improve the candidate selected, one can also select the antigen (or preferably the epitope) which is bound by more than one of the antibodies with the desired function. For example, if several of antibodies which kill tumor cells all bind to the same epitope, this will indicate that these are likely to be antibodies capable of protecting a broad patient population from such tumor. These antibodies can then be selected for further characterization and optimization; or, in the alternative, one can use the epitope in performing further screening for suitable candidates. These candidates can then be functionally screened and further characterized and optimized.

Examples of producing human monoclonal antibodies using the techniques of the invention are described below.

### EXAMPLE 1

### Human Antibodies Suitable for Cancer Tumor Therapy

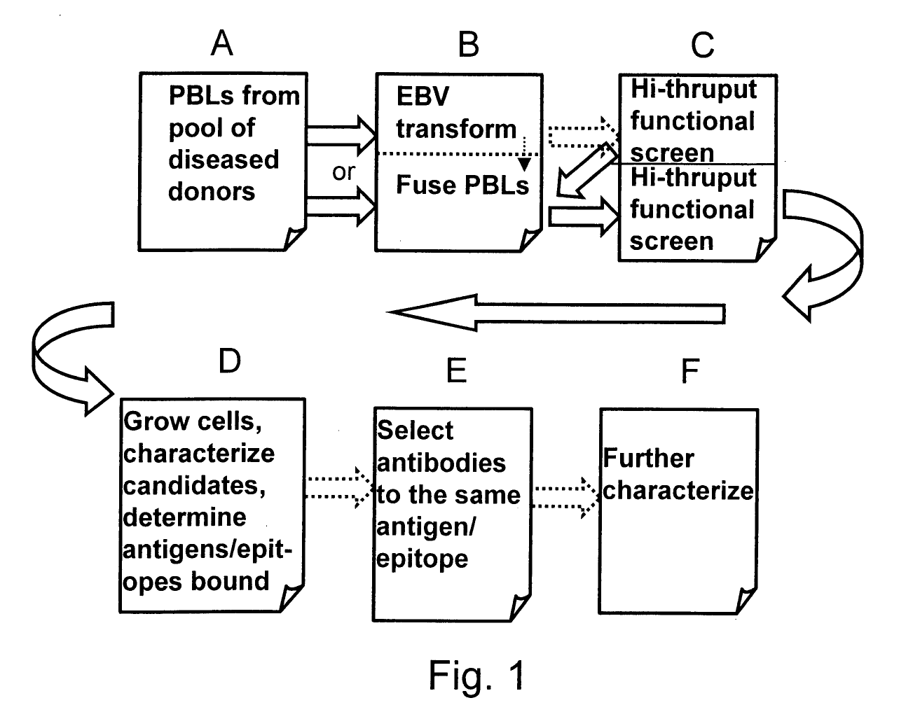

As shown in FIG. 1, panel A, to produce human antibodies suitable for tumor therapy with the methods of this invention, one establishes a pool of peripheral lymphocytes taken from a patient population all having a particular disease, e.g., a tumor or cancer. The lymphocytes are then EBV transformed and fused, or directly fused with a suitable cell line without EBV transformation, to immortalize them (panel B). Rather than fusing the EBV-transformed cells, one can perform a high throughput functional assay (as described above) to select cells producing antibodies that kill the tumor cells (indicated by the dotted arrows in panel B and the upper solid arrow from panel B to panel C). Following screening of EBV transformed cells, they are fused (arrow from panel C to B), or, for cells fused without EBV-transformation, they are screened (arrow from panel B to C).

These antibody-producing cells can then be grown, subcloned, and the antibodies further characterized and analyzed to determine their suitability for use as therapeutic candidates (panel D). One step in the characterization is to determine the epitopes bound by therapeutic candidates. Optionally, the antigen or epitope bound by more than one of the antibodies can be determined (indicated by the dotted arrow between panels D and E), and, optionally, subject to further analysis and characterization, including additional functional assays (panel F). Antibodies from a number of patients which all bind the same epitope, are more likely to be patient cross-protective.

One of the potential benefits of monoclonal antibodies with respect to the treatment of cancer is their ability to specifically recognize single antigens. It was thought that in some instances cancer cells possess antigens that were specific to that kind of transformed cell. It is now more frequently believed that cancer cells have few unique antigens, rather, they tend to over-express a normal antigen or express fetal antigens. Nevertheless, the use of monoclonal antibodies provided a method of delivering reproducible doses of antibodies to the patient with the expectation of better response rates than with polyclonal antibodies.

### EXAMPLE 2

### Finding Therapeutic Combinations of Antibodies

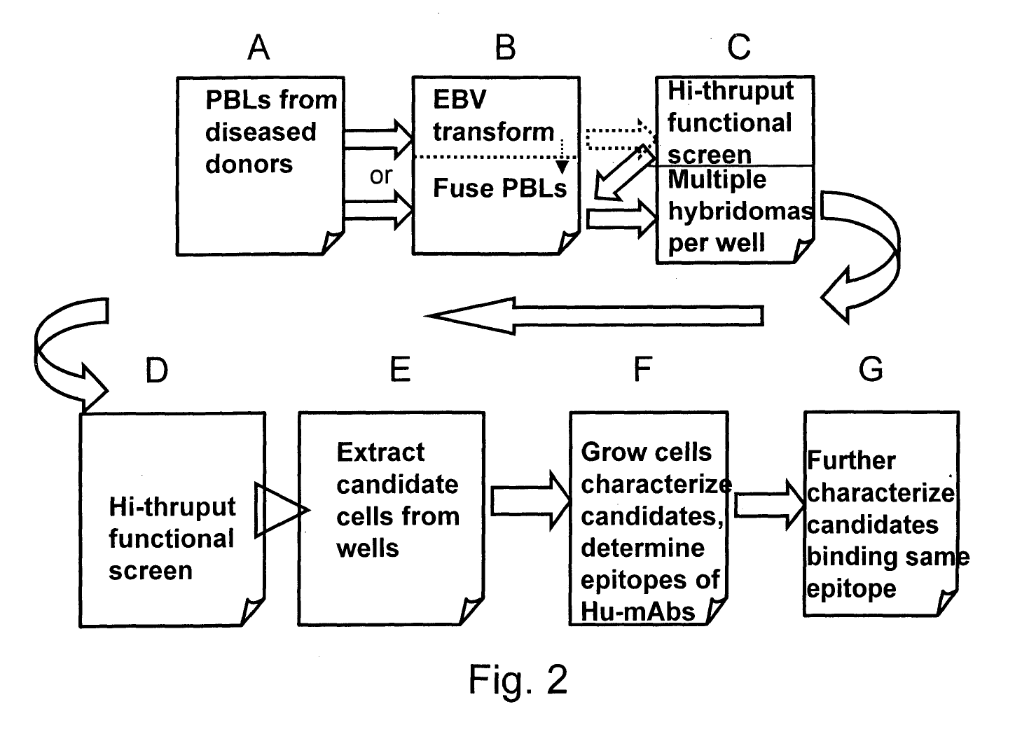

The methods described in Example 1 above can also be used to find combinations of human antibodies which act in an additive or synergistic manner. The primary difference here is that the when the antibody-producing cells are placed in the wells of a microtiter plate, they are not limit diluted as to where there is probably only one cell per well. Instead, they are limit diluted only to the point where there will likely be several cells per well. Following high throughput functional screening by any of the methods described above, samples are extracted from wells containing antibody-producing cells which induced a response in the target cells. The antibody-producing cells and the antibodies therein can then be further assayed and characterized to determine which are needed for the desired response in the target cells, whether more than one type of antibody is needed, and whether the desired response in the target cells can be induced more effectively with a plurality of antibodies than with any one antibody alone. This screening is illustrated in FIG. 2.

### EXAMPLE 3

### Isolating Therapeutic Antibodies Based on Their Interaction with Known Epitopes

Another method of isolating therapeutic human monoclonal antibodies would involve screening a library of human monoclonal antibodies against a library of peptides, and then isolating those antibodies which interact with peptides in the library known to represent or encompass disease-associated epitopes. As an example, human monoclonal antibody-producing cells suitable for tumor therapy are generated as in panels A to C of FIG. 1, and are then screened against a peptide library, e.g., a phage display peptide library. The cells producing antibodies which interact with peptides in the phage display library known to encompass or represent epitopes associated with tumors, based on available databases or independent research, are the ones of interest. Such antibody-producing cells can then be grown, subcloned, and the antibodies further characterized and analyzed to determine their suitability for use as therapeutic candidates (panel D of FIG. 1).

The terms and expressions above are exemplary and not limiting and the invention is defined only in the claims that follow and includes all equivalents of the subject matter of the claims.

What is claimed is:

1. A method of generating human monoclonal antibodies suitable as therapeutic candidates from a pool of human peripheral blood lymphocytes extracted from a number of individuals who have all been exposed to the same disease antigen, comprising:
immortalizing peripheral blood lymphocytes extracted from said individuals;
performing a high throughput functional screening assay of the immortalized lymphocytes against target cells associated with or affected by the disease, wherein the target cells are coated onto microbeads and placed in wells formed in one of the ends of a bundle of optical fibers, and wherein changes to the target cells resulting from the assay can be optically detected at the opposite end of the bundle;
selecting the immortalized lymphocytes producing human antibodies having specific, desired effects on the target cells, as determined by the assay; and
growing the selected lymphocytes and further characterizing the human antibodies produced by them to select therapeutic candidate antibodies.

2. The method of claim 1, wherein before the optical fiber screening assay, as an additional step, the immortalized lymphocytes are screened using a high-throughput screen which separates out antibody-producing cells.

3. The method of claim 2 wherein the high-throughput functional screening uses a flow cytometer system which allows determination of the source of a sample of antibody-producing cells.

4. The method of claim 1 wherein the optical fiber screening assay can be used to simultaneously detect different properties of different antibodies, wherein each such different antibody resides in a different well.

5. The method of claim 4 wherein the screening assay selects a plurality of antibodies which act together to produce a desired function or effect on the target cells.

6. The method of claim 2 wherein, before flow cytometry, the target cells and the immortalized lymphocytes are encapsulated in a manner such that changes in the target cells induced by the immortalized lymphocytes can be detected by flow cytometry.

7. The method of claim 1 further including isolating candidate antibodies derived from lymphocytes of different patients that bind to the sane antigens or epitopes.

8. The method of claim 7 comprising further characterizing the candidate antibodies selected based on their properties and predicted effects in vivo.

9. The method of claim 1 wherein high throughput functional screening selects for antibodies which kill the target cells, proliferate the target cells, affect target cell protein expression or intra-cellular signaling.

10. The method of claim 1 wherein the characterization of the therapeutic candidates is to determine those with high affinity, or high antibody expression rates or those which are likely to be most therapeutically effective.

11. The method of claim 1 wherein the high throughput functional screening selects for a plurality of antibodies which are effective in combination, or more effective in combination than individually.

12. The method of claim 2 wherein the lymphocytes are immortalized by EBV-transformation followed by fusion with a suitable myeloma cell, and flow cytometry is performed following the EBV immortalization and following the fusion.

13. The method of claim 1 wherein the subjects have tumors or cancers, the target cells are tumor cells common to some or all of the subjects and the high throughput functional screening selects for antibodies which kill the target cells.

14. The method of claim 1 further including the step of growing the selected lymphocytes and further characterizing the human antibodies produced by them to select therapeutic candidate antibodies.

15. A method of isolating therapeutic human monoclonal antibodies useful in therapy, comprising:
screening a library of human monoclonal antibodies or the cells producing them against a library of peptides;
isolating the antibodies and the cells producing them which interact with peptides in the library known to represent or encompass disease-associated epitopes.

16. The method of claim 15 wherein the library of human monoclonal antibodies is a tumor-associated library, and the peptides are known to represent or encompass tumor epitopes.

17. The method of claim 15 wherein the screening step is performed using a high throughput functional screening assay of the cells producing antibodies against target cells associated with or affected by the disease, wherein the target cells are coated onto microbeads and placed in wells formed in one of the ends of a bundle of optical fibers, and wherein changes to the target cells resulting from an assay performed in the wells can be optically detected at the opposite end of the optical fibers.
